(19) United States
(12) Patent Application Publication
(10) Pub. No.: US 2021/0330179 A1
(43) Pub. Date: Oct. 28, 2021

(54) ENDOSCOPE SYSTEM, CONTROL METHOD, AND COMPUTER READABLE RECORDING MEDIUM

(71) Applicant: OLYMPUS CORPORATION, Tokyo (JP)

(72) Inventor: Hidenori TSUBOI, Yokohama-shi (JP)

(73) Assignee: OLYMPUS CORPORATION, Tokyo (JP)

(21) Appl. No.: 17/367,825

(22) Filed: Jul. 6, 2021

Related U.S. Application Data
(63) Continuation of application No. PCT/JP2019/000379, filed on Jan. 9, 2019.

(51) Int. Cl. A61B 1/018; A61B 8/08; A61B 8/00

(52) U.S. Cl. CPC A61B 1/018; A61B 8/4483; A61B 8/4461; A61B 8/5207

(57) ABSTRACT

An endoscope system includes: an ultrasound transducer configured to output an electrical signal by receiving an ultrasound wave; an ultrasound observation apparatus configured to generate an ultrasound image based on a signal in a specific frequency band included in the electrical signal; a light source configured to supply illumination light to irradiate an inside of a subject; a light guide configured to guide the illumination light into the subject and scan an irradiation position of the illumination light into the subject along a specific trajectory according to an input drive signal; a driver configured to output the drive signal to the light guide; and a processor configured to control an operation of the driver to output the drive signal having a drive center frequency outside the specific frequency band to the light guide.

This application is a continuation of International Application No. PCT/JP2019/000379, filed on Jan. 9, 2019, the entire contents of which are incorporated herein by reference.

### BACKGROUND

The present disclosure relates to an endoscope system, a control method, and a computer readable recording medium.

In the related art, a direct view radial type ultrasound endoscope is known as an ultrasound endoscope including an insertion portion to be inserted into a subject (see, for example, Japanese Patent No. 4488203).

In the ultrasound endoscope described in Japanese Patent No. 4488203, the insertion portion includes a transducer, an illumination optical member, and an observation optical member.

The transducer includes a plurality of ultrasound transducers arranged along a circumferential direction surrounding a central axis of the insertion portion and configured to emit ultrasound waves. In addition, the transducer is provided with a through hole penetrating along the central axis of the insertion portion.

The illumination optical member includes an illumination lens, a light guide, and the like, and irradiates an inside of the subject with illumination light. The illumination optical member is inserted into the above-described through hole.

The observation optical member includes an objective lens, an imaging element, a video cable, and the like, and takes in the illumination light (hereinafter, referred to as return light) reflected from the subject. The observation optical member is inserted into the above-described through hole.

In addition, an optical fiber scanner is known that scans a light irradiation position along a specific trajectory according to an input drive signal (see, for example, International Publication No. 2017/195258).

For example, in the ultrasound endoscope described in Japanese Patent No. 4488203, if the optical fiber scanner described in International Publication No. 2017/195258 is inserted into the above-described through hole instead of the above-described illumination optical member and observation optical member, a diameter of the insertion portion may be reduced.

### SUMMARY

According to one aspect of the present disclosure, there is provided an endoscope system including: an ultrasound transducer configured to output an electrical signal by receiving an ultrasound wave; an ultrasound observation apparatus configured to generate an ultrasound image based on the electrical signal; a light source configured to supply illumination light to irradiate an inside of a subject; a light guide configured to guide the illumination light into the subject and scan an irradiation position of the illumination light into the subject along a specific trajectory according to an input drive signal; a driver configured to output the drive signal to the light guide; and a processor configured to control an operation of the driver, wherein the ultrasound observation apparatus is configured to generate the ultrasound image based on a signal in a specific frequency band included in the electrical signal, and the processor is configured to control the driver to output the drive signal having a drive center frequency outside the specific frequency band to the light guide.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an endoscope system according to a first embodiment;

FIG. 2 is a diagram illustrating a configuration of a distal end unit;

FIG. 3 is a perspective view illustrating a configuration of an optical fiber scanner;

FIG. 4 is a time chart explaining a control method executed by a processor;

FIG. 5 is a diagram illustrating a scanning trajectory of illumination light (irradiation position) scanned by the optical fiber scanner;

FIG. 6 is a graph explaining a drive center frequency of a drive signal to be output to a vibration unit;

FIG. 7 is a time chart explaining a control method according to a second embodiment; and

FIG. 8 is a diagram illustrating an endoscope system according to a third embodiment.

### DETAILED DESCRIPTION

Hereinafter, embodiments for carrying out the present disclosure (hereinafter referred to as embodiments) will be described with reference to the drawings. Note that the present disclosure is not limited by the embodiments described below. Further, in the description of the drawings, the same reference numerals are given to the same parts.

### First Embodiment

Schematic Configuration of Endoscope System

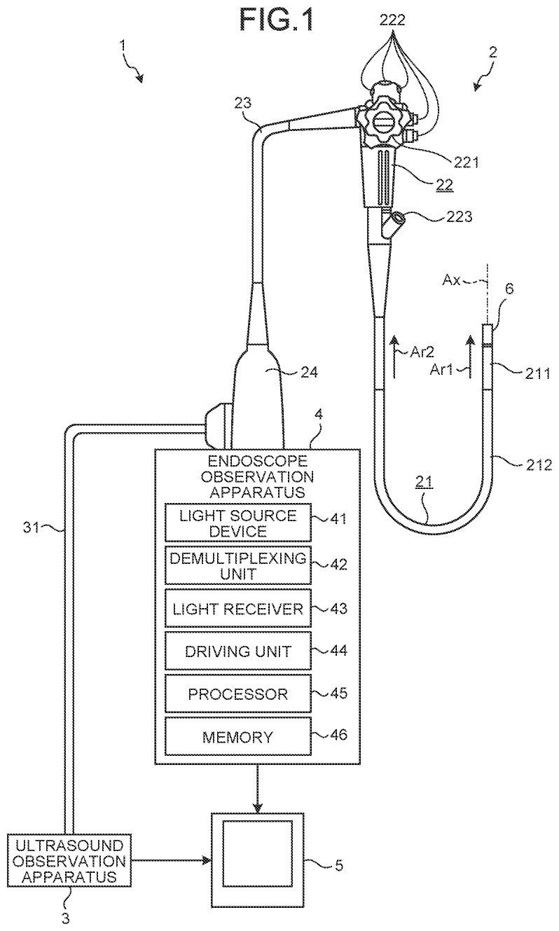

FIG. 1 is a diagram illustrating an endoscope system 1 according to a first embodiment.

The endoscope system 1 is a system that performs ultrasound diagnosis in a subject such as a human being by using an ultrasound endoscope. As illustrated in FIG. 1, the endoscope system 1 includes an ultrasound endoscope 2, an ultrasound observation apparatus 3, an endoscope observation apparatus 4, and a display device 5.

A part of the ultrasound endoscope 2 may be inserted into the subject. In addition, the ultrasound endoscope 2 has a function of outputting an echo signal (corresponding to an electrical signal according to the present disclosure) by transmitting an ultrasound pulse (acoustic pulse) toward a body wall in the subject and receiving an ultrasound echo (corresponding to an ultrasound wave according to the present disclosure) reflected from the subject, and a function of outputting illumination light (subject image) after taking in the illumination light reflected from the subject.

Note that a detailed configuration of the ultrasound endoscope 2 will be described later.

The ultrasound observation apparatus 3 is electrically connected to the ultrasound endoscope 2 via an ultrasound cable 31 (FIG. 1). The ultrasound observation apparatus 3 outputs a pulse signal to the ultrasound endoscope 2 via the ultrasound cable 31. In addition, the ultrasound observation apparatus 3 receives an echo signal from the ultrasound endoscope 2. Here, the ultrasound observation apparatus 3 generates an ultrasound image by performing predetermined processing on the echo signal.

A connector 24 (FIG. 1) to be described later of the ultrasound endoscope 2 is detachably connected to the endoscope observation apparatus 4. The endoscope observation apparatus 4 generates an endoscopic image based on a subject image output from the ultrasound endoscope 2.

Note that a detailed configuration of the endoscope observation apparatus 4 will be described later.

The display device 5 is configured using liquid crystal or organic electro luminescence (EL), and displays an ultrasound image generated by the ultrasound observation apparatus 3, an endoscopic image generated by the endoscope observation apparatus 4, or the like.

Configuration of Ultrasound Endoscope

Next, a configuration of the ultrasound endoscope 2 will be described.

As illustrated in FIG. 1, the ultrasound endoscope 2 includes an insertion portion 21, an operation unit 22, a universal cord 23, and the connector 24.

The insertion portion 21 is a portion to be inserted into the subject. Hereinafter, for convenience of description, one side (side of an distal end in an insertion direction into the subject) along a central axis Ax (FIG. 1) of the insertion portion 21 will be referred to as a distal end side Ar1, and another side (side of the operation unit 22 side) will be referred to as a proximal end side Ar2.

As illustrated in FIG. 1, the insertion portion 21 includes a distal end unit 6 located at an end portion on the distal end side Ar1, a curved portion 211 connected to the proximal end side Ar2 of the distal end unit 6 and capable of being curved, and a flexible tube 212 connected to the proximal end side Ar2 of the curved portion 211 and having flexibility.

Note that a detailed configuration of the distal end unit 6 will be described later.

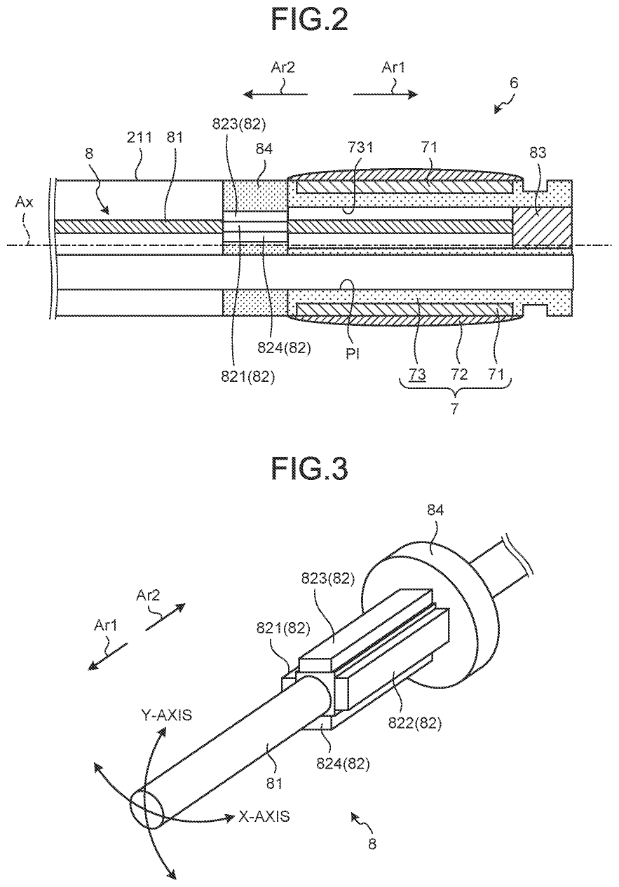

The operation unit 22 is connected to an end portion on the proximal end side Ar2 of the insertion portion 21 and receives various operations from a doctor or the like. As illustrated in FIG. 1, the operation unit 22 includes a bending knob 221 for bending the curved portion 211 and a plurality of operation members 222 for performing various operations. In addition, the operation unit 22 is provided with a treatment tool insertion port 223 communicating with a treatment tool pipeline PI (see FIG. 2) provided inside the insertion portion 21 (the distal end unit 6, the curved portion 211, and the flexible tube 212). When a treatment tool (not illustrated) such as a puncture needle is inserted from the treatment tool insertion port 223, the treatment tool passes through the treatment tool pipeline PI and then protrudes to an outside from an end surface on the distal end side Ar1 in the distal end unit 6.

The universal cord 23 is a cord that extends from the operation unit 22, and in which a transducer cable (not illustrated) for transmitting the above-described pulse signal or echo signal, a part of an optical fiber scanner 8 (see FIG. 2) constituting the distal end unit 6, and the like are disposed. Note that a part of the transducer cable and the optical fiber scanner 8 described above is also disposed inside the insertion portion 21 and the operation unit 22.

The connector 24 is provided at an end portion of the universal cord 23. Then, the connector 24 is connected to each of the ultrasound cable 31 and the endoscope observation apparatus 4.

Configuration of Distal End Unit

Next, a configuration of the distal end unit 6 will be described.

FIG. 2 is a diagram illustrating a configuration of the distal end unit 6. Specifically, FIG. 2 is a cross-sectional view of the distal end unit 6 taken along a plane including the central axis Ax.

As illustrated in FIG. 2, the distal end unit 6 includes a transducer 7 and the optical fiber scanner 8.

The transducer 7 is an electronic radial scanning transducer. That is, the transducer 7 transmits an ultrasound pulse in a direction orthogonal to the central axis Ax and transmits an ultrasound pulse in a rotation direction of 360° around the central axis Ax. As illustrated in FIG. 2, the transducer 7 is a unit in which a plurality of ultrasound transducers 71, an acoustic lens 72, and a backing material 73 are integrated, and has a substantially cylindrical shape coaxial with the central axis Ax.

The plurality of ultrasound transducers 71 is regularly arranged along a circumferential direction surrounding the central axis Ax. All of the plurality of ultrasound transducers 71 have the same shape, and each has a rectangular parallelepiped shape extending linearly along the central axis Ax. A pair of electrodes (not illustrated) is formed on an outside surface of the ultrasound transducer 71. The ultrasound transducer 71 converts a pulse signal input from the ultrasound observation apparatus 3 via the ultrasound cable 31, the transducer cable described above, and the pair of electrodes into an ultrasound pulse and transmits the ultrasound pulse into the subject. In addition, the ultrasound transducer 71 converts an ultrasound echo reflected from a portion to be observed in the subject into an electrical echo signal. The echo signal is input to the ultrasound observation apparatus 3 via the pair of electrodes, the transducer cable described above, and the ultrasound cable 31.

As illustrated in FIG. 2, the acoustic lens 72 has a substantially cylindrical shape whose outer peripheral surface is convexly curved, and constitutes an outer surface of the transducer 7. Then, the acoustic lens 72 has a function of converging the ultrasound pulse transmitted from the ultrasound transducer 71.

Note that, in the first embodiment, an acoustic matching layer for matching acoustic impedance of the ultrasound transducer 71 and the subject may be provided between the ultrasound transducer 71 and the acoustic lens 72.

As illustrated in FIG. 2, the backing material 73 is located on an inner side of the transducer 7 (a side close to the central axis Ax) with respect to the ultrasound transducer 71. In the first embodiment, the backing material 73 fills a cylindrical hollow portion constituted by the plurality of ultrasound transducers 71 so as to close the hollow portion. The backing material 73 has a function of attenuating unnecessary ultrasound vibration generated by the operation of the ultrasound transducers 71.

In the first embodiment, a through hole 731 (FIG. 2) penetrating from an end surface on the proximal end side Ar2 to the end surface on the distal end side Ar1 along the central axis Ax is formed in the backing material 73.

FIG. 3 is a perspective view illustrating a configuration of the optical fiber scanner 8. Specifically, FIG. 3 is a perspective view of the optical fiber scanner 8 as viewed from the distal end side Ar1.

The optical fiber scanner 8 corresponds to a light guide member according to the present disclosure. As illustrated in FIG. 2 or 3, the optical fiber scanner 8 includes a light guide path 81 and a vibration unit 82.

The light guide path 81 is, for example, a single-mode or multi-mode optical fiber. The optical fiber includes a core layer and a cladding layer. Light propagates in the core layer while being confined in the core layer.

The light guide path 81 is disposed in the following state.

The light guide path 81 is routed from the end portion on the proximal end side Ar2 to the end portion on the distal end side Ar1 inside the insertion portion 21. In addition, although not specifically illustrated, the end portion on the proximal end side Ar2 in the light guide path 81 is optically connected to the endoscope observation apparatus 4 when the connector 24 is attached to the endoscope observation apparatus 4. Further, as illustrated in FIG. 2, the distal end side Ar1 of the light guide path 81 protrudes toward the distal end side Ar1 in a cantilever shape with a light guide path fixing portion 84 as a fixed end, and is inserted into the through hole 731 in the backing material 73. In the light guide path 81, the end portion on the distal end side Ar1 from the light guide path fixing portion 84 is a free end. As a material constituting the light guide path fixing portion 84, the same material as the backing material 73 may be used, or a material different from the backing material 73 may be used.

In addition, in the through hole 731 of the backing material 73, the distal end side Ar1 is sealed by an optical system 83. The light guide path 81 introduces the illumination light supplied from the endoscope observation apparatus 4 into the subject and emits the illumination light from the end portion on the distal end side Ar1. The illumination light is emitted into the subject via the optical system 83. That is, the light guide path 81 corresponds to the light guide member body according to the present disclosure. In addition, the light guide path 81 takes in the illumination light (hereinafter, referred to as return light) reflected from the subject via the optical system 83 and guides the return light to the endoscope observation apparatus 4. Note that, as the light guide path 81, a plurality of optical fibers may be used in order to increase the efficiency of taking in the return light, or a multi-core type optical fiber may be used.

As described above, in the ultrasound endoscope 2 according to the first embodiment, the optical fiber scanner 8 is configured as a direct view type endoscope that observes a direction along the central axis Ax.

The vibration unit 82 is an actuator that generates vibration, and includes, for example, a piezoelectric actuator, an electromagnetic actuator, or an electrostatic actuator. In the first embodiment, as illustrated in FIG. 2 or 3, the vibration unit 82 includes four piezoelectric elements 821 to 824 disposed on an outside surface of the light guide path 81 at a position where the light guide path fixing portion 84 is provided. Note that in FIG. 3, a part of the light guide path fixing portion 84 is omitted. These four piezoelectric elements 821 to 824 are electrically connected to the endoscope observation apparatus 4 by electric wiring (not illustrated). Drive signals are input from the endoscope observation apparatus 4 to the four piezoelectric elements 821 to 824 via the electric wiring. Thus, each of the four piezoelectric elements 821 to 824 generates vibration.

For example, when a pair of the piezoelectric elements 821 and 822 facing each other among the four piezoelectric elements 821 to 824 generates vibration, the end portion on the distal end side Ar1 of the light guide path 81, which is a free end, resonantly vibrates and moves in an X-axis direction (FIG. 3). On the other hand, when another pair of the piezoelectric elements 823 and 824 facing each other generates vibration, the end portion on the distal end side Ar1 of the light guide path 81, which is a free end, resonantly vibrates and moves in a Y-axis direction (FIG. 3).

Then, the optical fiber scanner 8 scans the irradiation position of the illumination light into the subject along a specific trajectory according to the drive signals output from the endoscope observation apparatus 4.

Hereinafter, for convenience of description, among the drive signals output from the endoscope observation apparatus 4, the drive signal output to the pair of piezoelectric elements 821 and 822 is referred to as an X-axis drive signal, and the drive signal output to the pair of piezoelectric elements 823 and 824 is referred to as a Y-axis drive signal.

Configuration of Endoscope Observation Apparatus

As illustrated in FIG. 1, the endoscope observation apparatus 4 includes a light source device 41, a demultiplexing unit 42, a light receiver 43, a driving unit 44, a processor 45, and a memory 46.

The light source device 41 supplies illumination light for illuminating the inside of the subject to the light guide path 81.

The demultiplexing unit 42 guides the illumination light supplied from the light source device 41 to the end portion on the proximal end side Ar2 in the light guide path 81, and guides the return light emitted from the end portion on the proximal end side Ar2 in the light guide path 81 to the light receiver 43.

The light receiver 43 includes, for example, a lens, a detector, and the like. Then, the light receiver 43 outputs intensity information, to the processor 45, corresponding to the intensity of the return light that has been emitted from the end portion on the proximal end side Ar2 in the light guide path 81 and passed through the demultiplexing unit 42.

The driving unit 44 outputs a drive signal to the vibration unit 82.

The processor 45 is, for example, a central processing unit (CPU), a field-programmable gate array (FPGA), or the like, and controls the entire operation of the endoscope observation apparatus 4 according to a program (including a control program according to the present disclosure) stored in the memory 46. Note that detailed functions of the processor 45 will be described in “Control method executed by processor” described later.

The memory 46 stores a program executed by the processor 45 (including the control program according to the present disclosure), information necessary for processing of the processor 45, and the like.

Control Method Executed by Processor

Next, a control method executed by the processor 45 will be described.

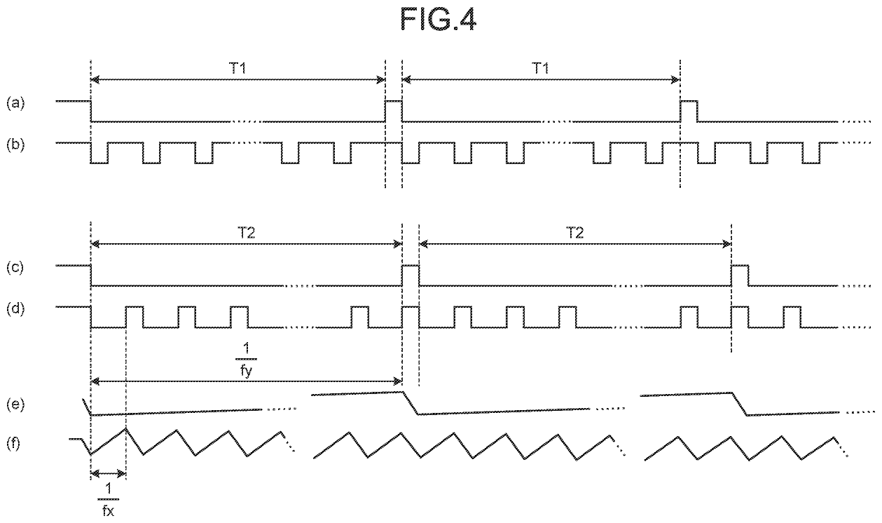

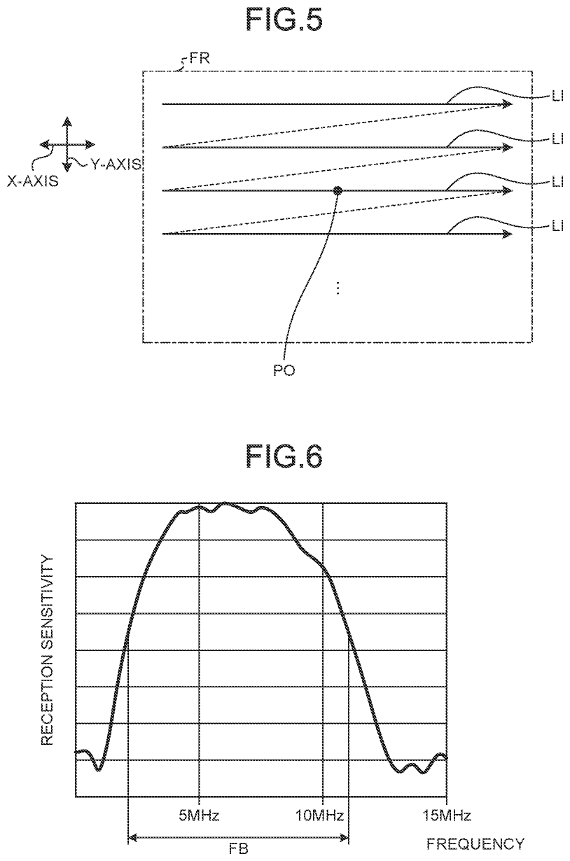

FIG. 4 is a time chart explaining the control method executed by the processor 45. Specifically, FIG. 4(a) illustrates a first frame synchronization signal for acquiring an ultrasound image for each frame. FIG. 4(b) illustrates a transmission pulse timing signal for outputting a pulse signal from the ultrasound observation apparatus 3. FIG. 4(c) illustrates a second frame synchronization signal for acquiring an endoscopic image for each frame FR (see FIG. 5). FIG. 4(d) illustrates a line synchronization signal for acquiring an endoscopic image for each horizontal line LI (see FIG. 5). FIG. 4(e) illustrates a signal waveform of the Y-axis drive signal. FIG. 4(f) illustrates a signal waveform of the X-axis drive signal.

The ultrasound observation apparatus 3 outputs a pulse signal to the ultrasound transducer 71 in a period during which the transmission pulse timing signal (FIG. 4(b)) is at a low level. In addition, the ultrasound observation apparatus 3 receives an echo signal from the ultrasound transducer 71 in a period during which the transmission pulse timing signal is at a high level. Then, the ultrasound observation apparatus 3 sequentially generates an ultrasound image of one frame every first period T1 (FIG. 4(a)) that is periodically repeated.

FIG. 5 is a diagram illustrating a scanning trajectory of illumination light (irradiation position) scanned by the optical fiber scanner.

On the other hand, the processor 45 controls the operation of the driving unit 44, outputs the Y-axis drive signal (FIG. 4(e)) to the pair of piezoelectric elements 823 and 824 in synchronization with the second frame synchronization signal (FIG. 4(c)), and outputs the X-axis drive signal (FIG. 4(f)) to the pair of piezoelectric elements 821 and 822 in synchronization with the line synchronization signal (FIG. 4(d)).

As a result, the end portion on the distal end side Ar1 of the light guide path 81, which is a free end, moves in the X-axis direction (FIG. 3) and the Y-axis direction (FIG. 3). Then, as illustrated in FIG. 5, the optical fiber scanner 8 repeats an operation of scanning the irradiation position of the illumination light into the subject by one horizontal line LI along the X-axis direction and then scanning by the next one horizontal line LI shifted in the Y-axis direction. That is, the optical fiber scanner 8 scans the irradiation position of the illumination light into the subject by so-called raster scanning.

Note that the scanning trajectory at the time of scanning the irradiation position of the illumination light to the inside of the subject is not limited to the trajectory illustrated in FIG. 5, and for example, other trajectories such as a spiral shape may be adopted. In other words, the Y-axis drive signal and the X-axis drive signal are not limited to the waveforms illustrated in FIGS. 4(e) and 4(f), and other waveforms may be adopted.

Here, when the above-described raster scanning is performed by the processor 45, the light receiver 43 sequentially outputs intensity information corresponding to the intensity of the return light taken in by the light guide path 81 and emitted from the end portion on the proximal end side Ar1 in the light guide path 81. In addition, the processor 45 sequentially stores, in the memory 46, related information in which the intensity information of the return light output from the light receiver 43 is associated with an XY coordinate value of an irradiation position PO corresponding to the position where the return light is taken in. The related information constitutes one pixel in an endoscopic image of one frame FR. Then, the processor 45 sequentially generates one endoscopic image of the one frame FR using the related information of the one frame FR every second period T2 (FIG. 4(c)) that is periodically repeated.

In the first embodiment, as illustrated in FIGS. 4(a) and 4(c), the first period Tl and the second period T2 are partially overlapped periods. That is, the period during which the ultrasound transducer 71 receives the ultrasound pulse and the period during which the driving unit 44 outputs the drive signal to the vibration unit 82 are partially overlapped.

Drive center frequency of drive signal output to vibration unit

Next, a drive center frequency of a drive signal output to the vibration unit 82 will be described.

FIG. 6 is a graph illustrating a drive center frequency of a drive signal output to the vibration unit 82. Specifically, FIG. 6 is a graph illustrating reception sensitivity of the ultrasound pulse in the ultrasound transducer 71, and the horizontal axis indicates a frequency and the vertical axis indicates the reception sensitivity. Note that the vertical axis indicates that the reception sensitivity is better as going upward, and the reception sensitivity is worse as going downward.

In the first embodiment, as illustrated in FIG. 6, the reception sensitivity of the ultrasound pulse in the ultrasound transducer 71 is low in a frequency band of less than 1 MHz and a frequency band of more than 13 MHz. Therefore, a frequency band of a signal used by the ultrasound observation apparatus 3 to generate an ultrasound image among signals of all frequencies included in an echo signal is set to a specific frequency band FB of 1 MHz or more and 13 MHz or less.

A drive center frequency fx (in FIG. 4(f)) of the X-axis drive signal and a drive center frequency fy (in FIG. 4(e)) of the Y-axis drive signal are set to frequencies outside the specific frequency band FB (frequency less than 1 MHz or more than 13 MHz). In the first embodiment, the drive center frequency fx of the X-axis drive signal and the drive center frequency fy of the Y-axis drive signal are set to frequencies (frequencies less than 1 MHz) lower than the specific frequency band FB.

The frequency band of the X-axis drive signal and the frequency band of the Y-axis drive signal may be frequency bands that do not overlap with the specific frequency band FB at all. Further, as long as the drive center frequencies fx and fy are frequencies outside the specific frequency band FB, a part of the frequency band of the X-axis drive signal and the frequency band of the Y-axis drive signal, and the specific frequency band FB may overlap each other.

According to the first embodiment described above, the following effects are obtained.

In the endoscope system 1 according to the first embodiment, the ultrasound transducer 71 and the optical fiber scanner 8 are provided integrally with the insertion portion 21. That is, the ultrasound transducer 71 and the optical fiber scanner 8 are disposed at positions close to each other.

Therefore, in the endoscope system 1 according to the first embodiment, the processor 45 controls the operation of the driving unit 44 and outputs drive signals having the drive center frequencies fx and fy outside the specific frequency band FB to the optical fiber scanner 8. The specific frequency band FB is a frequency band of a signal used by the ultrasound observation apparatus 3 to generate an ultrasound image among signals of all frequencies included in the echo signal.

Therefore, it is possible to reduce superimposition of noise caused by the drive signal on a signal used by the ultrasound observation apparatus 3 to generate an ultrasound image among echo signals, and deterioration of the ultrasound image may be suppressed.

Furthermore, the specific frequency band FB is set based on the reception sensitivity of ultrasound waves in the ultrasound transducer 71.

Therefore, it is possible to efficiently delete an unnecessary frequency band from frequency bands of the signal used by the ultrasound observation apparatus 3 to generate an ultrasound image.

The drive center frequency of the drive signal is lower than the specific frequency band FB.

Therefore, for example, the general-purpose piezoelectric elements 821 to 824 or the like may be adopted as the vibration unit 82, and the degree of freedom in design may be improved.

In addition, the optical fiber scanner 8 is adopted as the light guide member according to the present disclosure.

Therefore, it is possible to effectively reduce the diameter of the insertion portion 21.

### Second Embodiment

Next, a second embodiment will be described.

In the following description, the same reference numerals are given to the same configurations as those of the above-described first embodiment, and the detailed description thereof will be omitted or simplified.

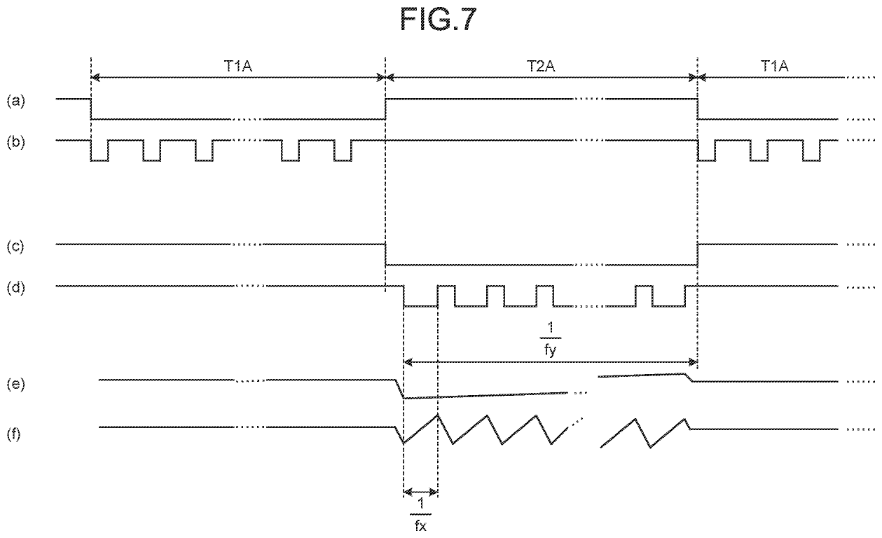

FIG. 7 is a time chart explaining a control method according to a second embodiment. Specifically, FIG. 7 is a diagram corresponding to FIG. 4. That is, FIG. 7(a) illustrates the first frame synchronization signal. FIG. 7(b) illustrates the transmission pulse timing signal. FIG. 7(c) illustrates the second frame synchronization signal. FIG. 7(d) illustrates the line synchronization signal. FIG. 7(e) illustrates the signal waveform of the Y-axis drive signal. FIG. 7(f) illustrates the signal waveform of the X-axis drive signal.

As illustrated in FIG. 7, the second embodiment is different from the first embodiment in the control method executed by the processor 45.

Specifically, the ultrasound observation apparatus 3 according to the second embodiment sequentially generates an ultrasound image of one frame in each first period T1A of a first period T1A (FIG. 7(a)) and a second period T2A (FIG. 7(a)) that are alternately repeated, similarly to the first embodiment described above.

On the other hand, the processor 45 according to the second embodiment sequentially generates an endoscopic image of one frame FR in each second period T2A, similarly to the above-described first embodiment.

That is, in the second embodiment, the period during which the ultrasound transducer 71 receives the ultrasound pulse and the period during which the driving unit 44 outputs the drive signal to the vibration unit 82 do not overlap at all.

In the second embodiment, two types of periods, which are the first and second periods T1A and T2A, are provided, and the first and second periods T1A and T2A are alternately repeated, but the present disclosure is not limited thereto. For example, a configuration may be adopted in which three or more types of periods (including the first and second periods T1A and T2A) that do not overlap each other at all may be provided, and the three or more types of periods may be sequentially repeated.

According to the second embodiment described above, the following effects are obtained.

In the second embodiment, the period during which the ultrasound transducer 71 receives the ultrasound pulse and the period during which the driving unit 44 outputs the drive signal to the vibration unit 82 do not overlap at all.

Therefore, noise caused by the drive signal is not superimposed on the echo signal, and deterioration of the ultrasound image may be effectively suppressed.

Since noise caused by the drive signal is not superimposed on the echo signal, the drive center frequencies fx and fy may be frequencies within the specific frequency band FB or frequencies outside the specific frequency band FB.

### Third Embodiment

Next, a third embodiment will be described.

In the following description, the same reference numerals are given to the same configurations as those of the above-described first embodiment, and the detailed description thereof will be omitted or simplified.

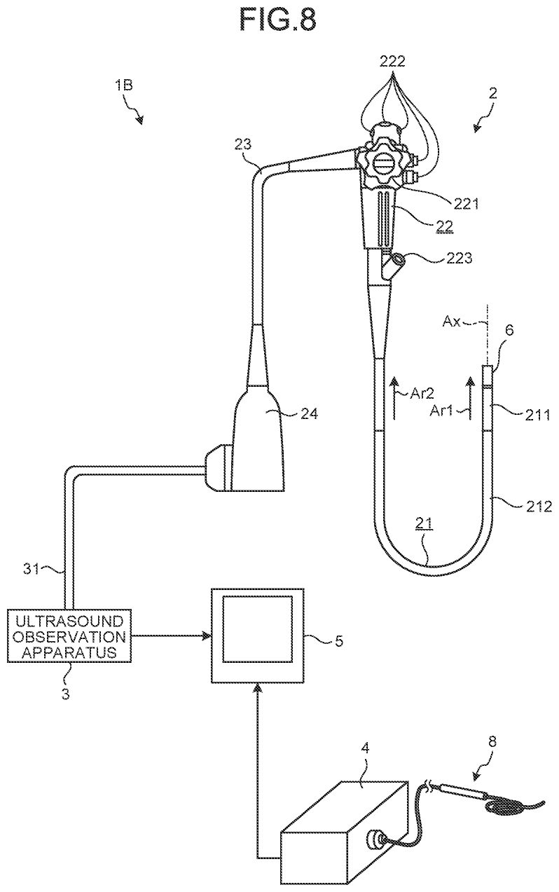

FIG. 8 is a diagram illustrating an endoscope system 1B according to the third embodiment. Specifically, FIG. 8 is a diagram corresponding to FIG. 1.

In the endoscope system 1 according to the first embodiment described above, the optical fiber scanner 8 is provided integrally with the ultrasound endoscope 2 (insertion portion 21).

On the other hand, in the endoscope system 1B according to the third embodiment, as illustrated in FIG. 8, the optical fiber scanner 8 is provided separately from the ultrasound endoscope 2 and is connected to the endoscope observation apparatus 4. Note that the ultrasound endoscope 2 according to the third embodiment is not provided with the optical fiber scanner 8 described in the first embodiment described above. In addition, a connector 24 according to the third embodiment is connected only to the ultrasound cable 31.

The optical fiber scanner 8 according to the third embodiment is inserted into the treatment tool pipeline PI from the treatment tool insertion port 223, and is used in a state where the distal end portion protrudes to the outside from the end surface on the distal end side Ar1 of the distal end unit 6.

Note that the control method executed by the processor 45 is a control method similar to that in the first embodiment described above. In addition, a control method similar to the control method described in the second embodiment described above may be used.

According to the third embodiment described above, the following effects are obtained.

The optical fiber scanner 8 according to the third embodiment is inserted into the treatment tool pipeline PI from the treatment tool insertion port 223, and is used in a state where the distal end portion protrudes to the outside from the end surface on the distal end side Ar1 of the distal end unit 6. That is, also in the endoscope system 1B according to the third embodiment, similarly to the endoscope system 1 according to the first embodiment described above, the ultrasound transducer 71 and the optical fiber scanner 8 are disposed at positions close to each other.

Therefore, by adopting a control method similar to that of the first embodiment described above or a control method similar to that of the second embodiment described above as a control method executed by the processor 45, effects similar to those of the first embodiment described above or the second embodiment described above are obtained.

### OTHER EMBODIMENTS

Although the embodiments for carrying out the present disclosure have been described so far, the present disclosure should not be limited only by the above-described first to third embodiments.

In the above-described first to third embodiments, an electronic radial scanning transducer is adopted as the transducer 7, but the present disclosure is not limited thereto, and an electronic convex scanning transducer may be adopted. Further, the present disclosure is not limited to an electronic scanning method, and a mechanical scanning method may be adopted.

In the above-described first and second embodiments, the optical fiber scanner 8 is configured as a direct view type endoscope that observes a direction along the central axis Ax. However, the present disclosure is not limited thereto, and the optical fiber scanner 8 may be configured as a side view type endoscope that observes a direction orthogonal to the central axis Ax or an oblique view type endoscope that observes a direction intersecting the central axis Ax.

In the above-described first to third embodiments, the optical fiber scanner 8 scans the illumination light (irradiation position) two-dimensionally in the X-axis direction and the Y-axis direction, but the present disclosure is not limited thereto, and scanning may be performed only one-dimensionally in the X-axis direction or the Y-axis direction.

In the above-described first to third embodiments, the optical fiber scanner 8 is adopted as the light guide member according to the present disclosure, but the present disclosure is not limited thereto, and an optical crystal that changes a refractive index according to an input drive signal (according to application of a voltage) may be adopted. That is, the optical crystal makes it possible to scan the illumination light (irradiation position) along a specific trajectory by changing the refractive index.

In the above-described first to third embodiments, the light guide path for irradiating the inside of the subject with the illumination light and the light guide path for taking in the return light are configured by the common light guide path 81, but the present disclosure is not limited thereto, and may be configured by separate light guide paths.

In the above-described first to third embodiments, the specific frequency band according to the present disclosure may be a band that is within the frequency band FB and narrower than the frequency band FB. In this case, the drive center frequency of the drive signal may be a frequency within the frequency band FB as long as the drive center frequency is outside the narrowed band.

According to the endoscope system, the control method, and the computer readable recording medium storing the control program, it is possible to suppress deterioration of an ultrasound image.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general concept as defined by the appended claims and their equivalents.

What is claimed is:

1. An endoscope system comprising:
an ultrasound transducer configured to output an electrical signal by receiving an ultrasound wave;
an ultrasound observation apparatus configured to generate an ultrasound image based on the electrical signal;
a light source configured to supply illumination light to irradiate an inside of a subject;
a light guide configured to guide the illumination light into the subject and scan an irradiation position of the illumination light into the subject along a specific trajectory according to an input drive signal;
a driver configured to output the drive signal to the light guide; and
a processor configured to control an operation of the driver, wherein
the ultrasound observation apparatus is configured to generate the ultrasound image based on a signal in a specific frequency band included in the electrical signal, and
the processor is configured to control the driver to output the drive signal having a drive center frequency outside the specific frequency band to the light guide.

2. The endoscope system according to claim 1, further comprising an insertion portion adapted to be inserted into the subject, the insertion portion including a treatment tool pipeline through which a treatment tool is inserted and through which the inserted treatment tool protrudes to an outside, wherein
the ultrasound transducer is provided integrally with the insertion portion, and
the light guide is provided separately from the insertion portion and is inserted into the treatment tool pipeline.

3. The endoscope system according to claim 1, further comprising an insertion portion adapted to be inserted into the subject, wherein
each of the ultrasound transducer and the light guide is provided integrally with the insertion portion.

4. The endoscope system according to claim 1, wherein the specific frequency band is set based on reception sensitivity of an ultrasound wave in the ultrasound transducer.

5. The endoscope system according to claim 1, wherein the drive center frequency is lower than the specific frequency band.

6. The endoscope system according to claim 1, wherein the light guide includes
a light guide body configured to guide the illumination light into the subject and irradiate the inside of the subject with the illumination light, and
a vibrator configured to scan an irradiation position of the illumination light into the subject along the specific trajectory by vibrating the light guide body according to the input drive signal.

7. An endoscope system comprising:
an ultrasound transducer configured to output an electrical signal by receiving an ultrasound wave;
an ultrasound observation apparatus configured to generate an ultrasound image based on the electrical signal;
a light source configured to supply illumination light to irradiate an inside of a subject;
a light guide configured to guide the illumination light into the subject and scan an irradiation position of the illumination light into the subject along a specific trajectory according to an input drive signal;
a driver configured to output the drive signal to the light guide; and
a processor configured to control an operation of the driver, wherein
the ultrasound transducer is configured to receive an ultrasound wave during a first period of the first period and a second period that are repeated, and
the processor is configured to control the driver to output the drive signal to the light guide during the second period.

8. An endoscope system comprising:
an insertion portion adapted to be inserted into a subject;
an ultrasound transducer configured to output an electrical signal by receiving an ultrasound wave;
an ultrasound observation apparatus configured to generate an ultrasound image based on the electrical signal;
a light source configured to supply illumination light to irradiate an inside of the subject;
a light guide configured to guide the illumination light into the subject and scan an irradiation position of the illumination light into the subject along a specific trajectory according to an input drive signal;
a driver configured to output the drive signal to the light guide; and
a processor configured to control an operation of the driver, wherein
each of the ultrasound transducer and the light guide member is provided integrally with the insertion portion,
the ultrasound observation apparatus is configured to generate the ultrasound image based on a signal in a specific frequency band included in the electrical signal, and
the processor is configured to control the driver to output the drive signal having a drive center frequency outside the specific frequency band to the light guide.

9. The endoscope system according to claim 8, wherein the specific frequency band is set based on reception sensitivity of an ultrasound wave in the ultrasound transducer.

10. The endoscope system according to claim 8, wherein the drive center frequency is lower than the specific frequency band.

11. The endoscope system according to claim 8, wherein the light guide includes
a light guide body configured to guide the illumination light into the subject and irradiate the inside of the subject with the illumination light, and
a vibrator configured to scan an irradiation position of the illumination light into the subject along the specific trajectory by vibrating the light guide body according to the input drive signal.

12. A control method comprising
scanning an irradiation position of illumination light into a subject along a specific trajectory by controlling a driver to output a drive signal to a light guide, wherein
a drive center frequency of the drive signal is a frequency outside a specific frequency band, and
the specific frequency band is a frequency band of a signal used by an ultrasound observation apparatus to generate an ultrasound image, among signals of all frequencies included in an electrical signal output by an ultrasound transducer upon receiving an ultrasound wave.

13. A control method comprising
scanning an irradiation position of illumination light into a subject along a specific trajectory by controlling a driver to output a drive signal to a light guide during a second period other than a first period in which an ultrasound transducer receives an ultrasound wave, of the first period and the second period that are repeated.

14. A non-transitory computer-readable recording medium on which an executable program is recorded, the program causing a processor of a computer to execute:
scanning an irradiation position of illumination light into a subject along a specific trajectory by controlling a driver to output a drive signal to a light guide, wherein
a drive center frequency of the drive signal is a frequency outside a specific frequency band, and
the specific frequency band is a frequency band of a signal used by an ultrasound observation apparatus to generate an ultrasound image, among signals of all frequencies included in an electrical signal output by an ultrasound transducer upon receiving an ultrasound wave.

15. A non-transitory computer-readable recording medium on which an executable program is recorded, the program causing a processor of a computer to execute:
scanning an irradiation position of illumination light into a subject along a specific trajectory by controlling a driver to output a drive signal to a light guide during a second period other than a first period in which an ultrasound transducer receives an ultrasound wave, of the first period and the second period that are repeated.
